(19) United States
(12) Patent Application Publication
(10) Pub. No.: US 2003/0180372 A1
(43) Pub. Date: Sep. 25, 2003

(54) Preparation of immune goat colostrum against rotavirus and its use for prevention of rotavirus induced diarrhoea in infants and young children

(76) Inventors: Shobhana Dattatraya Kelkar, Pune (IN); Rejendra Prabhakar Deolankar, Pune (IN)

Correspondence Address: VENABLE, BAETJER, HOWARD AND CIVILETTI, LLP, P.O. BOX 34385, WASHINGTON, DC, 20043-9998, US

(21) Appl. No.: 10/100,165

(22) Filed: Mar. 19, 2002

(51) Int. Cl. A61K 39/15; A61K 35/20; C12N 7/00; C12N 7/01

(52) U.S. Cl. 424/535; 424/215.1; 435/235.1

(57) ABSTRACT

The present invention relates to a colostrum and to a process for the preparation thereof to prevent rotavirus induced diarrhoea of G1 to G4, G5 and G6, G8 to G10. The process comprises in mixing the rotavirus specimens from infected children with an adjuvant to obtain water in oil emulsions. The virus singularly or the emulsion is inoculated into pregnant goats. The colostrum is collected from the immunized goats after delivery, dried and added to a carrier.

### FIELD OF INVENTION

This invention relates to the preparation of immune goat colostrum against rotavirus and its use for prevention of rotavirus induced diarrhoea in infants and young children.

### BACKGROUND OF THE INVENTION

The diet and nutrition of infants in the age group of 3 to 30 months is of vital importance in deciding their susceptibility to common infections. Though the most traditional as well as packaged baby foods provide adequate nutrition to the infant, they do not address their resistance to common viral and bacterial infections. One of the major causes of mortality and morbidity among children of this age group, especially in developing countries, is rotaviral infections leading to acute diarrhoea followed by severe dehydration.

In the developed countries, 90-95% of rotaviruses in stool specimens could be serotyped as G1-G4. However, in the developing countries a large number of specimens remain untypeable. Besides, many mixed serotypes are detected. This indicates the activity of human rotavirus serotypes other than G1-G4, affecting children in the developing countries, making the prevention of Rotavirus diarrhoea more difficult.

Rotavirus induced diarrhoea cannot be controlled by antibiotics. It is believed that rotavirus associated diarrhoea is unlikely to be controlled through improved sanitation, water supply or hygiene.

Rotavirus vaccine trials have given inconsistent results in developing countries. Moreover, vaccine was withdrawn in USA because of suspected vaccine-induced complications like intussusceptions in children.

Alternative approach for the prevention of rotavirus diarrhoea in children is passive immunity provided by the artificial feeding of antibody containing preparations.

### PRIOR ART

The concept of imparting a prophylactic activity (against pathogenic organisms) to processed infant foods is not much exploited. Rotavirus is an exception, which has shown some favourable results. Initially, the Nestle Research Centre at Lausanne in Switzerland reported work on using bovine milk immunoglobulins for conferring passive immunity to infants (Brussow et al., 1987, Hilpert et al., 1987). Subsequently, passive immunization of children with bovine colostrum containing antibodies to rotavirus was proposed by Davidson et al., 1989. Both studies used the serotypes G1 to G4, which are prevalent in Europe. Guario et al.,1994 reported the anti rotavirus effect of human serum immunoglobulin administered severally. Children who received these immunoglobulin had significantly faster recovery with education in the duration of diarrhoeal virus excretion. The immunoglobulins of bovine colostrum provide the major antimicrobial protection and confer a passive immunity to newborn animals, until its own immune system matures. The concentration in colostrum of specific antibodies against pathogens can be prepared by immunizing cows with these pathogens or their antigens. Immune milk products are preparations made of such hyper immune colostrum or antibodies enriched from it. These preparations can be used to give effective specific protection against different enteric diseases. Colostral immunoglobulin supplements designed for farm animals are commercially available in many countries (Korhonen et al., 2000).

It was shown experimentally that passively acquired antibodies from either serum or colostrum can protect young animals against diarrhoea caused by rotavirus infection (Snodgrass DR and Wells PW, 1978). This passive protection can be exploited, by stimulating dams to produce in her colostrum and milk high titres of antibodies to rotavirus.

In temperate countries, rotavirus associated diarrhoea is an endemic problem among low birth weight babies, high risk infants in hospitals, infants in orphanages etc. A randomized trial of oral immunoglobulin prepared against human rotavirus in these babies infected with rotavirus was carried out (Ebina et al., 1983). Orally administered bovine rota colostrum significantly protected 5 out of 6 infants from diarrhoea caused by rotavirus but not from natural in apparent infection.

The dose schedule to prepare rota colostrum was as follows: Holstein cows were immunized subcutaneously with Wa strain of human rotavirus (HRV) G1 serotype (105.5 TCID 50/ml×5 ml) 4 times at 10 days intervals until calving, first with complete adjuvant and three times without adjuvant. Neutralizing antibody titer of colostrum, which was used for prevention of diarrhoea, was 1:400-1280. Colostrum was administered in children as follows: 20 cc colostrum with 1:1280 titer daily per child for one month and then colostrum with 1:400 titer for 10 days.

In 1987, a group of scientists from Nestle Research Centre, Lausanne, Switzerland and universities in Germany immunized cows with four HRV serotypes and isolated bovine milk immunoglobulin with potent antiviral activity, suitable for inducing passive immunity to infantile rotavirus gastroenteritis (Brussow et al., 1987).

The dose schedule consisted of6 doses, one every week and variety of routes of inoculation viz. s/c, intracistemal, i/m.i/v etc. The resultant product, colostrum had 100 fold increase in neutralizing milk antibody titers over those of controls and 10 times higher than those in commercial pooled immunoglobulin concentrate.

Australian scientists prepared bovine colostrum by vaccinating pregnant cows intramuscularly six times during the last trimester of their pregnency (Davidson et al., 1989). The rotavirus vaccine containing all four HRV serotypes was formalin inactivated and emulsified with equal volume of Freunds incomplete adjuvant. The first does consisted of 10 ml of vaccine. The second dose contained, in addition, 10 ml of inactivated UK bovine rotavirus and was given 14 days later. After further 14 days, and weekly thereafter on three occasions each cow was given 10 ml of vaccine containing only inactivated rotavirus SA-11 (serotype 3).

The virus neutralizing antibody titer was greater than 1/12800 for all serotypes in the final preparation given in children. They have also shown the efficacy of a 10 day course of bovine colostrum with high antibody against all four HRVs in protecting children from rotavirus diarrhoea. Nine of 65 controls and one of 55 treated children acquired rotavirus infection. Bovine milk immunoglobulin fed to children during the course of the rotavirus gastroenteritis reduced the duration of rotavirus excretion. This shortening of duration of rotavirus excretion may also help in reducing the likelihood or rotavirus cross infections and the secondary attack rate.

Ebina et al (1992) demonstrated that rota colostrum derived from HRV immunized cows could prevent gastroenteritis in a murine model of HRV infection. Protection against disease correlated with the in vitro neutralizing activity of cow colostrum against the MO strain. However, purified IgG or IgM and secretary IgA fractions derived from rota colostrum did not prevent rotavirus induced diarrhoea. Purified Igs seem to be inactivated in the gastro duodenal tracts. In colostrum, IgG and IgA seem to be protected by other milk proteins. Authors have also confirmed the efficacy of the colostrum among children from a baby care centre. Oral administration of rota colostrum for two weeks could prevent rotavirus associated diarrhoea.

To see whether human serum immunoglobulin is effective in acute rotavirus gastroenteritis a prospective, double blind placebo controlled study was preformed (Guarino et al., 1994). A single oral dose of 300 mg/kg of body weight of human serum immunoglobulin was administered. Children who received immunoglobulin had significantly faster clinical improvement of clinical condition and stool pattern than control children. Mean total duration of rotaviral diarrhoea and hospital stay was reduced among children who received treatment of human serum immunoglobulin. Neutralizing titer of human immunoglobulin preparation was between 1:800-1:3200 for rotavirus major serotypes viz. G1-G4.

Bovine colostrum based immune milk products have proven effective in prophylaxis against various infectious diseases. Good results have been obtained with products targeted against rotavirus. The products have been evaluated for potential as dietary supplements and in clinical nutrition.

Successful treatment of rotavirus diarrhoea in children with hyper immune bovine colostrum has been reported. (Mitra et al 1995 Sarkar et al 1998), Ylitalo et al 1998).

However, there are no reports on preparation of goat colostrum against human rotaviruses, causing gastroenteritis among infants, though WO 98/54226, dated Dec. 3, 1998 by Hodgkinson A J discloses the preparation of immunoglobulin A by immunizing ewes with a commercially available vaccine, 3K Scourguard containing, bovine rotavirus and other antigens and had claimed that goat be selected as mammal for immunization.

Thus, the prior art is essentially cocord with serotypes G1 to G4 for preparation of a bovine serum. The prior art is not concerned with nor discloses the serotypes of rotavirus G5, G6 and G8 to G10. Further, even if the prior art was concerned with the serotypes of rotavirus G5, G6 and G8 to G10 it could not be used for preparation of bovine serum as known with the rotavirus GI to G4. As cow not immune to the rotavirus G5, G6 and G8 to G10.

### OBJECTS OF THE INVENTION

An object of the present invention is to prepare a process for the preparation of a plurality of colostrums against the wide varieties of rotavirus G5, G6 and G8 to G10 and as well G1 to G4.

A further object of the present invention is to propose a nutritional supplement containing colostrum for new born human babies so that the adsorption of colostrum is must faster before the gut closure phenomenon occurs.

Still further object of the invention is to prepare rota colostrum from pregnant goats as it is cost effective and has nutritional advantages over cow milk

Yet another object of the invention is to prepare rota colstrum from pregnant goats and which may effectively be used for the treatment of rotavirus G5, G6 and G8 to G10 and as well as G1 and G4.

### DESCRIPTION OF INVENTION

According to this invention there is provided a process for preparing colostrum to prevent rotavirus induced diarrhoea comprising collecting rotavirus specimens from infected children, mixing the said virus with an adjuvant to obtain water in oil suspension; innoculating said virus and as adjuvant mixture at multiple sites by intravenous route at different intervals for immunization; collecting the colostrum samples from said immunized goats after delivery; drying the colostrum samples to obtain dried powder thereof.

The adjuvant employed in the present invention is Freund's complete/incomplete adjuvant Head killed Mycobacterium tuberculoses, 60 to 70 mgs emulsified in 50.0 ml of mineral oil. Thus is done using a pestle and mortar to grind up the bacteria in a small volume of oil before the remainder of the oil is added. The use of Freund's complete adjuvant is effective in stimulating cell mediated immunity and potentiates the production of certain immunoglobulins. The incomplete Freund's Adjuvant does not contain mycobacteris.

Reference to the numerical values hereinabove with respect to the adjuvant is only by way of example and without intending to imply any limitation.

### EXAMPLE 1

Selection of Rotavirus Serotypes

Group A rotavirus specimens collected from 446 hospitalized children from Pune, India wer characterized for G serotypes. It was found that majority of the specimens which could be serotyped belonged to G1 and G2 serotypes.

Preparation of Virus Stocks

Lyophilized stocks of G1 (KU strain) and G2 (S2 strain) were grown in MA 104 cell line, with 5% fatal bovine serum and confirmed as rotavirus. The viruses were titrated after 3-4 passages in the cell line. The viruses were checked for their identity as G1 and G2 rotavirus serotypes when sufficient titre was attained. ELISA using monoclonal antibodies (MAbs) obtained from Dr. Shazo Urasawa, Sapporo, Japan was used for serotyping. RNA PAGE was also carried to confirm long and short RNA pattern for G1 and G2 serotypes respectively.

Preparation of the Rotaviruses on Mass Scale

For inoculation of one dose of rotavirus in 10 goats, at least a virus stock of one burler (two liter roller bottle) was prepared. There was complete cytopathic effect within 18-24 hrs. The bottles were frozen at −70° C. and thawed twice under tap water, and the material was collected. The virus titre was again determined. Before using the stock for immunization, the virus stock was centrifuged at 4000 rpm for 30 mins to get rid of cell debris and stored at 70° C. till further use.

Rotavirus Antibody Titres in Unimmunized Goats:

Rotaviruses are common pathogens for goats and therefore goat milk/colostrum shows appreciable natural ‘N’ antibody titers against rotaviruses. Before immunization, we determined background ELISA antibody and neutralizing 9N) antibody titres in serum and colostrum of unimmunized goats. For this, a group of 10 pregnant goats were selected. The goats were already given number by ear tagging on the goat farm. They were bled & serum was separated. After the delivery, colostrum was collected and stored frozen at −20° C. The serum/colostrum samples collected from each goat was given NIV specimen number.

Immunization of Goats

For immunization, different schedules were selected and were numbered by capital letters like schedule A,B,C,D etc. Each schedule was designed with specific question in mind.

For each schedule, generally 4-5 goats were selected. Thus, 43 goats were used for 9 schedules viz. A,B,C,D,E,F,G,H and I. And 10 goats as unimmunised controls. The information regarding breed age weight, parity status and litter size of the goats was collected.

TABLE I
Immunization Schedule

| | NO. OF | | | | ROUTE/NO. | DELIVERY |
|---|---|---|---|---|---|---|
| SCHEDULE | GOATS | DOSE | DAY | INOCULUM | OF SITES | DAY (AVG) |
| A | 5 | 1 | 0 | HRV-1 + IFA | IM/4 | 24-30 |
| Adjuvant | | 2 | 15 | HRV-1 + IFA | IM/4 | |
| mixed | | | | | | |
| manually | | | | | | |
| | | 3 | 23 | HRV-1 + IFA + | IM/4 | |
| | | | | SA-11 | | |
| B | 5 | 1 | 0 | HRV-1 + IFA | IM/4 | 24-30 |
| Adjuvant | | 2 | 15 | HRV-1 + IFA | IM/4 | |
| mixed with | | | | | | |
| waring | | | | | | |
| blender | | | | | | |
| | | 3 | 23 | HRV-1 + IFA + | IM/4 | |
| | | | | SA-11 | | |
| C | 5 | 1 | 0 | HRV-1 | IV | 23-27 |
| | | 2 | 12 | HRV-1 | IV | |
| | | 3 | 20 | HRV-1 + IFA + | IM/4 | |
| | | | | SA-11 | | |
| D | 5 | 1 | 0 | HRV-1 | IV | 23-26 |
| | | 2 | 12 | HRV-1 | IV | |
| | | 3 | 20 | HRV-1 + | IM/4 | |
| | | | | SA-11 | | |
| E | 4 | 1 | 0 | HRV-1 + CFA | IM/4 | 32-36 |
| | | 2 | 21 | HRV-1 + IFA | IM/4 | |
| | | 3 | 33 | HRV-1 + | IM/4 | |
| | | | | SA-11 | | |
| F | 4 | 1 | 0 | HRV-1 + CFA | IM/4 | 34-38 |
| | | 2 | 21 | HRV-1 + IFA | IM/4 | |
| | | 3 | 33 | HRV-1 | IM/4 | |
| G | 5 | 1 | 0 | HRV-1 + IFA | IM/4 | 40-47 |
| | | 2 | 15 | HRV-1 + IFA | IM/4 | |
| | | 3 | 930 | HRV-1 + IFA + | IM/4 | |
| | | | | SA-11 | | |
| H | 5 | 1 | 0 | HRV-2 + IFA | IM/4 | 40-48 |
| | | 2 | 15 | HRV-2 + IFA | IM/4 | |
| | | 3 | 30 | HRV-2 + IFA + | IM/4 | |
| | | | | SA-11 | | |
| I | 5 | 1 | 0 | ISOLATE + IF | IM/4 | 40-46 |
| | | | | A | | |
| | | 2 | 15 | ISOLATE + IF | IM/4 | |
| | | | | A | | |
| | | 3 | 30 | ISOLATE + IF | IM/4 | |
| | | | | A + SA-11 | | |

Field Work was Carried out and Consisted of,

Bleeding of goats for serum samples. Milk samples (if available) from goats before immunization schedule were also collected. Sera were separated.

Mixing of virus and the adjuvant to get water in oil suspension. For this work, a wooden hood with ultraviolet tube was transported to goat and arranged in a room close to the goat farm. The virus was taken on wet ice in heavily insulated thermos flask.

Inoculation of virus-adjuvant mixture or virus alone either by IM (intramuscular) route near the lymph nodes at multiple sites or virus without adjuvant by I.V. (intravenous route).

Three doses of the virus were inoculated at different intervals as shown in the Table 1.

Collection of the colostrum samples: Before the goats delivered, sterile bottles were kept ready to collect colostrum samples as required. Newborn goat kids were fed first and only remaining available colostrum was collected. After the immunization schedules, the goats delivered within one weeks time. The staff of the goat farm collected colostrum samples on day 1,2,3 and 4 post delivery (PD) day and milk on 10 PD day. The samples were stored at −20° C. at the farm. NIV received telephonic message regarding deliveries of the goats. The colostrum samples were transported to NIV by the laboratory staff.

When the samples were received at NIV, 15.0 ml of each sample was taken out and processed on the same day to avoid repeated freezing and thawing of the samples. Colostrum samples of the goats, belonging to one schedule were pooled. Colostrum pools of 1 st PD day 2, 3, 4th PD day and pool of milk obtained on 10th PD day were prepared. Thus, three pools was prepared from one schedule a sample of each colostrum pool was also stored at NIV.

Spray Drying of Colostrum Samples:

The colostrum pools were spray dryed, ELISA antibody and neutralising (‘N’) antibody titres in dried powders of colostrum were determined and compared with titres in the colostrum pools of different schedules.

Processing of Colostrum Samples for Testing ELISA Ab and Neutralizing (N) Antibody Titres:

Since colostrum samples of first 4 days after delivery are very viscous they cannot be directly used in ELISA test or in tissue culture for ‘N’ test.

Colostrum samples were processed on the same day, when they were brought from goat farm to the laboratory.

For processing, about 15.0 ml of colostrum samples were centifuged at 2500 rpm for half hour. The middle clear layer was obtained and it was re-centrifuged at 16000 rpm for one hour. Again, middle layer, which was quite clear, was obtained. It was divided into 5 aliquots and stored in cryobox at −20° C. Subsequently, it was used for testing antibodies against immunizing rotavirus. It was also tested in ‘N’ test for antibodies to heterologous virus.

Testing the Colostrum Samples for Antibodies Against Rotavirus:

This was done by Two Methods:

ELISA antibody was tested by indigenously developed ELISA. ELISA was carried out on serially diluted colostrum samples. This test detects total antibody to rotaviruses in the samples.

Colostrum samples were tested for ‘N’ antibody with starting dilution of 1:50 ‘N’ antibody to rotavirus is usually directed to VP7, which is a glycoprotein on the outermost capsid of the virus. Antibodies raised against VP7 are specific for different serotypes of rotavirus. When the virus is inoculated into animals, a small amount of heterologous or cross-reactive antibodies against VP4 protein are also raised. These both types of ‘N’ antibodies are very important from point of view of protection against rotavirus. Development of heterologous antibody against other rotavirus serotypes was also measured. Colostrum samples were also checked for the presence of rotavirus antigen by ELISA.

Results:

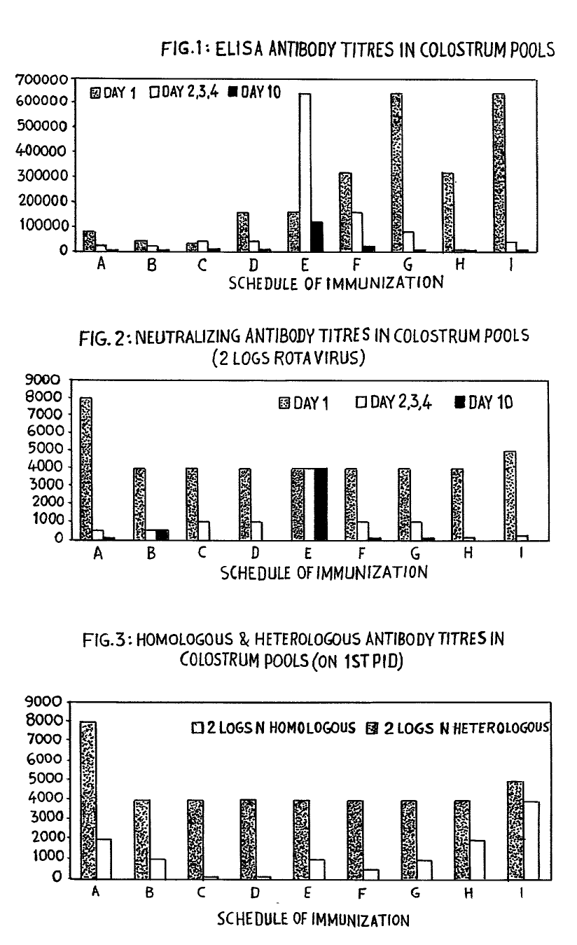

The results of ELISA and ‘N’ antibody levels in colostrum pools and spray dried power prepared employing different immunization schedules are presented in details in Table II-IV and graphically in FIGS. 1, 2 and 3 ‘N’ antibody titre in colostrums samples show in table 3 were determined on stored colostrum at −20° C. for 2-4 months till spray dried colostrum was obtained. Titres in FIG. 2 are of freshly collected colostrum.

The results are expressed as highest dilution of colostrum sample that shows presence of ELISA antibody or which neutralizes 2 log/1 log of virus.

High level of homologous and heterologous antibody was developed against human rotavirus serotypes 1 and 2 (G1 and G2 serotypes). Antibody titres were retained fairly well during the spray drying procedure.

Rotavirus/Antigen was not detected in any of the colostrum samples.

TABLE II
ELISA Antibody titres in
Colostrum pools/powders of different schedules

| | | | ELISA Titres in |
|---|---|---|---|
| | Day of Post | ELISA Titres | dried powder |
| Schedule | delivery pool | in colostrums | (1% trehalose)* |
| A | Day 1 | 80000 | 40000 |
| | Day 2, 3, 4 | 20000 | 10000 |
| | Day 10 | 1000 | 500 |
| B | Day 1 | 40000 | 20000 |
| | Day 2, 3, 4 | 20000 | 10000 |
| | Day 10 | 1000 | 500 |
| C | Day 1 | 32000 | 16000 |
| | Day 2, 3, 4 | 40000 | 20000 |
| | Day 10 | 5000 | 2500 |
| D | Day 1 | 160000 | 80000 |
| | Day 2, 3, 4 | 40000 | 10000 |
| | Day 10 | 5000 | 2000 |
| E | Day 1 | 600000 | 400000 |
| | Day 2, 3, 4 | 400000 | 100000 |
| F | Day 1 | 600000 | 400000 |
| | Day 2, 3, 4 | 100000 | 40000 |
| G | Day 1 | 800000 | 200000 |
| | Day 2, 3, 4 | | |
| H | Day 1 | 600000 | 100000 |
| I | Day 1 | 800000 | 200000 |

*Trehalose used as stabilizer
**ND: not done

TABLE III
Rotavirus neutralising (‘N’) antibody titres in colostrum
pools/dried powder of different schedules

| | | ‘N’ Ab Titre (HRV-1) | | ‘N’ Ab Titre in dried | |
|---|---|---|---|---|---|
| | | in colostrum | | powder *1% Trehalose | |
| | Days of Post | 2 Logs | | 2 Logs | |
| | delivery | Virus | 1 Log Virus | Virus | 1 Log Virus |
| Schedule | pool | ‘N’ | ‘N’ | ‘N’ | ‘N’ |
| A | Day 1 | 8000 | >32000 | >1000 | 12000 |
| | Day 2, 3, 4 | 500 | >32000 | ND | ND |
| | Day 10 | 100 | >2000 | ND | ND |
| B | Day 1 | 4000 | >32000 | >1000 | 12000 |
| | Day 2, 3, 4 | 500 | >32000 | ND | ND |
| | Day 10 | 500 | >2000 | ND | ND |
| C | Day 1 | 1000 | 2000 | -ve | 100 |
| | Day 2, 3, 4 | 1000 | >32000 | ND | ND |
| | Day 10 | -ve | 2000 | ND | ND |
| D | Day 1 | 4000 | 32000 | -ve | 100 |
| | Day 2, 3, 4 | 1000 | 16000 | ND | ND |
| | Day 10 | -ve | 2000 | ND | ND |
| E | Day 1 | 2000 | 6000 | 2000 | 6000 |
| | Day 2, 3, 4 | 1000 | 4000 | 500 | 1000 |
| F | Day 1 | 2000 | 5000 | 1000 | 2000 |
| | Day 2, 3, 4 | 500 | 1000 | 200 | 500 |
| G | Day 1 | 3000 | 7000 | 1500 | 3000 |
| | Day 2, 3, 4 | 500 | 1000 | 100 | 100 |
| H | Day 1 | 2000 | 8000 | 800 | 2000 |
| | Day 2, 3, 4 | 100 | 500 | 100 | 500 |
| I | Day 1 | 2000 | 3000 | 1000 | 2000 |
| | Day 2, 3, 4 | 100 | 500 | ND | ND |

TABLE IV
Homologous and Heterologous “N” antibody titres in colostrum pools of different
schedule on 1^{st} post delivery day

| | | HRV-1 | | HRV-2 | | HRV-3 | | HRV-4 | |
|---|---|---|---|---|---|---|---|---|---|
| | Immunising | 2 logs | 1 log | 2 logs | 1 log | 2 logs | 1 log | 2 logs | 1 log |
| Schedule | virus | “N”* | “N” | “N” | “N” | “N” | “N” | “N” | “N” |
| A | HRV-1 | 8000 | 32000 | 2000 | 3000 | 6000 | 6000 | 4000 | 12000 |
| B | HRV-1 | 4000 | 32000 | 1000 | 1000 | 4000 | 4000 | 2000 | 2000 |
| C | HRV-1 | 4000 | 4000 | 100 | 100 | 6000 | 8000 | 100 | 1000 |
| D | HRV-1 | 4000 | 16000 | ND | ND | 3000 | 7000 | 100 | 800 |
| E | HRV-1 | 4000 | 4000 | 1000 | 1000 | 4000 | 6000 | 2000 | 6000 |
| F | HRV-1 | 4000 | 4000 | ND- | ND- | 6000 | | 1000 | 3000 |
| G | HRV-1 | 4000 | 8000 | 1000 | 2000 | 4000 | 6000 | 6000 | 6000 |
| H | HRV-2 | 4000 | 6000 | 4000 | 6000 | 1000 | 6000 | 4000 | 8000 |
| I | HRV-2 | 4000 | >8000 | 5000 | >8000 | 6000 | 6000 | 1000 | 8000 |
| | like | | | | | | | | |
| | NIV | | | | | | | | |
| | isolate: | | | | | | | | |
| | 958644 | | | | | | | | |

“N” = Neutralising/Neutralisation

In general, human rotavirus is a good immunogen. High titred antibody to human rotavirus was developed by all the immunization schedules, which reflects that rota colostrum has been successfully raised among pregnant goats. High titre antibody was developed on 1st PD day and then it declined.

It was noted that schedule D developed good antibody response without adjuvant and hence it is cost effective. Secondly, schedule E in which CFA and IFA was used in 1st and 2nd doses respectively, the antibody titres seemed to retain for 10 days time. The number of goats in this schedule was insufficient.

All the first day colostrum pools were tested for the detection of rotavirus by ELISA and found to be negative.

Spray dried powder of colostrum samples retained high titres of ELISA and ‘N’ antibody. Important observation is, if the goats are healthy, the antibody titres against rotavirus are high. Conclusions:

1) High titered rota colostrum could be produced in goat breeds viz cross bred (B×S) and indigenous goats.

2) No live rotavirus could be detected in colostrums preparations. Base level ELISA antibody titres among unimmunised goat colostrum was about 1:110,000 whereas among immunised goats it was about 32,000-6,40,000 ‘N’ antibody titres were negligible in colostum from unimmunised goats, where as high levels of ‘N’ antibody titres were observed among immunized goats.

3) There is less heterologous antibody response in Schedule B as compared to Schedule A (Table IV). This may be because virus and adjuvant mixing was done by waring blender, instead of manual mixing which resulted in destruction of VP4 the protein responsible for inducing heterologous antibody response.

4) Schedule C & D (i/v two doses followed by 3rd i/m dose) did not show good heterologous antibody response.

5) Schedule E seemed to be best schedule. However, colostrum from only one goat were available. Hence, it is to be repeated. Schedule F was without booster dose. In spite of that, high levels of antibodies to G3 virus were detected. This observation needs further study.

6) Among schedules G, H & I and I schedule gave the best homologous as well as heterologous response. Thus, NTV isolate, 958644 may be used for preparing high titred colostrum against G1 as well as G2 serotypes which are most common in India.

7) Immune response to human rotavirus strains viz. HRV-1 (G1), HRV-2 (G2) and Indian isolate of rotavirus was satisfactory.

### EXAMPLE 2

Transfer of immunity against rotavirus from immunized mother goats to their kids.

Among goats, transplacental immunity is absent. Placental layers do not allow antibody to be transferred to kids. Hence, antibody levels among goat kids will be present only as a result of suckling. Maternal antibodies secreted in the colostrum are transmitted rapidly to the circulation of new born animal across the walls of digestive tract.

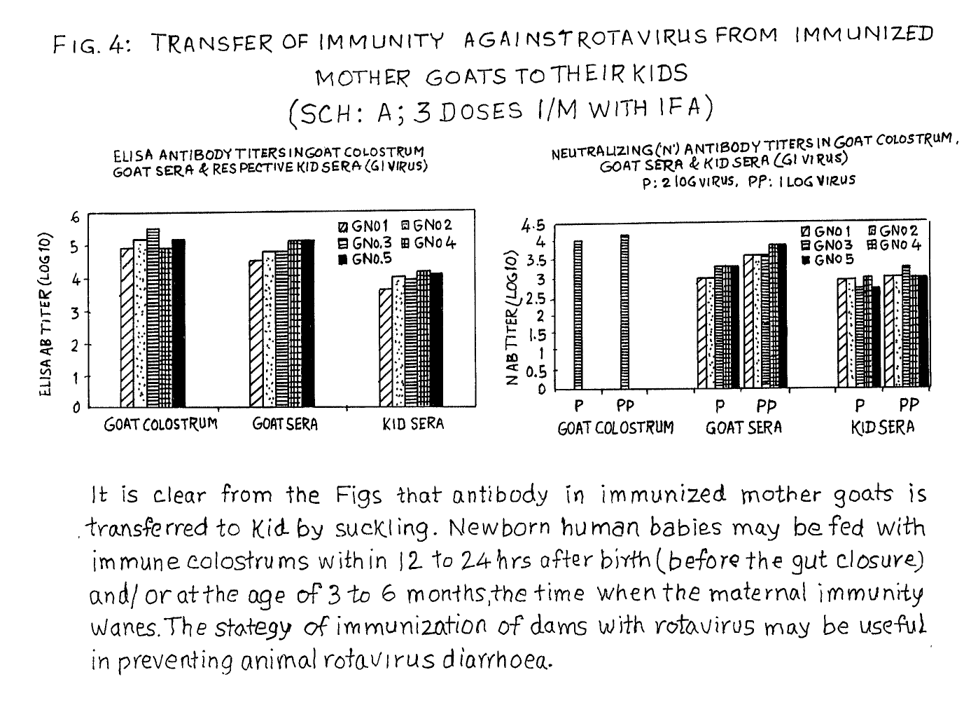

Kids born to immunised mothers and suckled for 4 days were bled and the sera samples were tested for the presence of ELISA antibody ‘N’ antibody titres. Further, 10 kids were bled at the age of one year to evaluate whether rotavirus antibodies in kids are retained for the period of one year. The results provided are in table V and VI and FIG. 4.

TABLE V
ELISA Antibody Titres in Goats and their kids
(Goat kids suckled to immunised mothers)

| | | ELISA Titres of mother goats | | | ELISA Titres of Goat Kids | | |
|---|---|---|---|---|---|---|---|
| | | | | Post | | | Titre |
| Sr. | | Goat | Pre Sera | delivery | Goat | Titre, 4 days | after one |
| No. | Schedule | No. | Titre | sera Titre | Kid No. | after birth | year |
| 1 | A | 9605 | 1:500 | 1:128,000 | 30145 | 1:12,000 | 1:2000 |
| | | | | | 30146 | >1:16,000 | |
| 2 | A | 9613 | 1:1000 | 1:32,000 | 30116 | 1:4000 | — |
| | | | | | 30117 | 1:4000 | 1:1000 |
| 3 | A | 9622 | 1:2000 | 1:64.000 | 30128 | 1:10,000 | — |
| | | | | | 30129 | 1:10,000 | |
| 4 | A | 9624 | 1:2000 | 1:64,000 | 30114 | 1:8,000 | |
| | | | | | 30115 | 1:8,000 | |
| 5 | A | 40106 | 1:1000 | 1:128,000 | 30134 | 1:16,000 | |
| | | | | | 30136 | 1:16,000 | |
| 6 | B | 9607 | 1:1000 | 1:128,000 | 30132 | 1:10,000 | |
| | | | | | 30133 | 1:4,000 | |
| 7 | B | 9623 | 1:500 | 1:64,000 | 30148 | >1:16,000 | |
| 8 | B | 9626 | 1:1000 | 1:64,000 | 30111 | 1:2,000 | |
| | | | | | 30112 | 1:8,000 | 1:1000 |
| | | | | | 30113 | 1:10,000 | 1:2000 |
| 9 | B | 40136 | 1:2000 | 1:28,000 | 30147 | 1:12,000 | |
| 10 | B | 40365 | 1:1000 | 1:16,000 | 30118 | 1:8,000 | |
| | | | | | 30119 | 1:10,000 | |
| 11 | C | 10241 | 1:1000 | 1:32,000 | 30143 | 1:6000 | 1:1000 |
| 12 | C | 10274 | 1:2000 | 1:8000 | 30123 | 1:1000 | |
| 13 | C | 28671 | 1:1000 | 1:4000 | 30137 | 1:10,000 | |
| | | | | | 30138 | 1:12,000 | |
| 14 | D | 9645 | 1:1000 | 1:32,000 | 30142 | 1:2000 | 1:1000 |
| 15 | D | 9654 | 1:100 | 1:16,000 | 30139 | 1:4000 | 1:2000 |
| | | | | | 30140 | 1:1000 | 1:4000 |
| 16 | D | 40302 | 1:1000 | 1:2000 | 30125 | 1:1000 | |
| | | | | | 30126 | — | |
| 17 | D | 40380 | 1:1000 | 1:4000 | 30120 | 1:10,00 | |
| | | | | | 30121 | 1:2000 | |
| | | | | | 30122 | 1:1000 | |
| 18 | E | 40350 | 1:1000 | 1:128,000 | | | |
| 19 | E | 40358 | 1:500 | 1:128,000 | Kid | 1:1000 | |
| 20 | E | 35150 | 1:1000 | 1:128,000 | 30/54 | >1:16000 | |
| 21 | F | 28607 | 1:1000 | 1:128,000 | 30/60 | >1:16000 | |
| 22 | F | 28608 | 1:500 | 1:128,000 | 30/58 | >1:16000 | |

TABLE VI
Neutralizing (N) Antibody Titres in Goats and their Kids
(Goat Kids suckled to immunised mothers)

| | | | “N” Titres of Mother Goats | | “N” Titres of Goat Kids | |
|---|---|---|---|---|---|---|
| Sr. | Mother | | | Post Delivery | Titre 4 Days | Titre After |
| No. | No. | Schedule | Pre Sera | Sera | After Birth | One Year |
| 1 | 9605 | A | P*-ve PP**-ve | P2000, PP8000 | P500, PP1000 | P-ve, PP-ve |
| 2 | 9613 | A | P-ve, PP50 | P1000, PP4000 | P800, PP1000 | P-ve, PP-ve |
| | | | | | P800, PP1000 | P-ve, PP-ve |
| 3 | 9622 | A | P-ve, PP50 | P2000, PP8000 | P > 2000, PP > 2000 | P-ve, PP-ve |
| | | | | | P1000, PP > 2000 | — |
| 4 | 9624 | A | P-ve, PP-ve | P2000, PP4000 | P500, PP > 2000 | NA |
| | | | | | P800, PP2000 | — |
| 5 | 40106 | A | P-ve, PP-ve | P2000, PP8000 | P > 1000, PP > 1000 | NA |
| 6 | 9607 | B | P-ve, PP-ve | P1000, PP8000 | P500, PP1000 | NA |
| | | | | | P > 2000, PP > 2000 | — |
| | | | | | P500, PP1000 | — |
| 7 | 9623 | B | P-ve, PP-ve | P100, PP1000 | P100, PP1000 | NA |
| 8 | 9626 | B | P-ve, PP-ve | P1000, PP8000 | P800, PP2000 | P-ve, PP-ve |
| | | | | | P200, PP2000 | — |
| | | | | | P1800, PP > 2000 | — |
| 9 | 40136 | B | P-ve, PP-ve | P500, PP2000 | P100, PP100 | |
| 10 | 40365 | B | P-ve, PP-ve | P100, PP8000 | P1800, PP > 2000 | NA |
| | | | | | P > 2000, PP > 2000 | — |
| 11 | 10241 | C | P-ve, PP-ve | P100, PP100 | P-ve, PP-ve | P-ve, PP-ve |
| 12 | 10274 | C | P-ve, PP-ve | P500, PP2000 | P400, PP400 | NA |
| 13 | 28671 | C | P-ve, PP-ve | P-ve, PP8000 | P400, PP1000 | NA |
| 14 | 9645 | D | P-ve, PP-ve | P100, PP100 | P-ve, PP-ve | P-ve, PP-ve |
| 15 | 9654 | D | P-ve, PP-ve | P100, PP1000 | P-ve, PP-ve | P-ve, PP-ve |
| | | | | | P-ve, PP-ve | P-ve, PP-ve |
| 16 | 40302 | D | P-ve, PP-ve | P-ve, PP2000 | P-ve, PP100 | NA |
| | | | | | P200, PP200 | — |
| 17 | 40380 | D | P-ve, PP-ve | P-ve, PP8000 | P400, PP2000 | — |
| | | | | | P400, P > 500 | — |
| | | | | | P800, PP2000 | NA |
| 18 | 40350 | E | | | P2000, PP2000 | NA |
| 19 | 40358 | E | | P100, PP500 | P-ve, PP-ve | |
| 20 | 35150 | E | | P500, PP1000 | P1000, PP2000 | NA |
| 21 | 28607 | F | P-ve, PP-ve | P500, PP500 | P2000, PP2000 | NA |
| 22 | 28608 | F | P-ve, PP-ve | P500, PP1000 | P > 2000, PP > 2000 | NA |

Neutralization of 2 Logs of Virus is Indicated as ‘P’

‘N’ antibody titre of 1:128 to rotavirus serotypes is protective against that particular serotype (ref. Fields Virology). As seen from the results, most of the newborn kids had protective antibodies against immunising serotype in their blood. However, ELISA and neutralizing antibody titer was not retained after one year of age in goat kids. But, the results are very encouraging. This strategy of immunising mothers with required rotavirus diarrhoea in several animals. This is an important finding, which has emerged from the present invention.

### EXAMPLE3

Goats were artificially bred by synchronisation of estrus and thereafter artificial insemination (A1).

There is certain advantage of A1, which predicts fairly accurate date of delivery, so that immunisation of goats can be planned well. However, there are some disadvantages, the (I) success rate of A1 is about 70-80% (ii) cost of A1 is about Rs. 500/goat, which is rather high (iii) expertise for carrying out successful A1 is not easily available.

Considering above issues and because goats for which A1 was done, were not available, we planned the experiment employing naturally bred goats.

The major disadvantage of naturally bred goats is to predict the exact date of ovulation heat period. However, unlike cows, the goats are seasonal breeders and hence appropriate dates for heat period can be ascertained. Thus, induction of the some flexibility in immunisation may permit production of hyperimmune colostrum in the naturally bred nannies and thereby saving the cost of A1 and requirement of expertise.

Selection of Rotavirus Strains:

Goats were immunised with HRV-4 virus and boosting with G6 (bovine Lincoln) strain. Accordingly, virus stocks of HRV-4 and G6 (Bovine Lincoln) viruses were prepared and the virus was titrated).

Immunisation Schedules:

1) Since ‘E’ schedule employing complete Freund's adjuvant (CFA) followed by incomplete Freund's adjuvant (IFA) and last dose of virus without adjuvant gave the best results last year, it was decided to use ‘E’ schedule with rotavirus serotypes G1 & G4, using schedules J & K respectively.

2) This year we also included intranasal immunization schedule L. After 2-3 doses of virus (NIV isolate 958644) by intranasal route, the ELISA antibody titre was very low. Therefore, 4th dose was inoculated along with incomplete Freund's adjuvant by intramuscular route.

3) The details of schedules J,K,L, are shown in table VII.

TABLE VII
IMMUNISATION SCHEDULE

| | No. | | | | | Delivery |
|---|---|---|---|---|---|---|
| Sched- | of | | | | Route/No. | Day |
| ule | Goats | Dose | Day | Inoculum | of Sites | (Avg.) |
| J | 4 | 1 | | HRV-1 + CFA | IM/4 | |
| | | 2 | | HRV-1 + IFA | IM/4 | |
| | | 3 | | HRV-1 + SA-11 | IM/4 | |
| K | 5 | 1 | 0 | HRV-4 + CFA | IM/4 | 36 |
| | | 2 | 21 | HRV-4 + IFA | IM/4 | |
| | | 3 | 30 | HRV-4 + BOV | IM/4 | |
| | | | | LIN | | |
| L | 6 | 1 | 0 | 958644 | Intranasal | 36 |
| | | 2 | 21 | 958644 | Intranasal | |
| | | 3 | 30 | 958644 | Intranasal | |

Results

Schedule J: One goat delivered after 1 st dose and one after 2nd doses. The immunization schedule of three doses could not be completed. The ‘N’ antibody titers after two was 1:1000.

Remaining two were not pregnant therefore additional two doses were given during their next pregnancy. The titres of ‘N’ Antibodies were between 1:3000 to 1:6000.

Schedule K: ELISA and ‘N’ antibody titres in the colostrum pools/dried powder against HRV-4 virus is presented in tables VIIIa & VIIIb. As experienced last year, with ‘E’ schedule ‘N’ antibody titres were retained fairly well. ‘N’ antibody titres against boosting animal virus viz. Bovine Lincoln virus are yet to be carried out.

Schedule L (intranasal): As seen in the tables Ixa and Ixb, ELISA and neutralizing antibody titres seem to be low for above schedule. One reason could be, we have estimated IgG antibody only. Intranasal is likely to develop more of IgA antibodies, which are more important for protection against diarrhoea however we have no test for determining IgA antibodies in goats. To determine IgA levels, we need to raise anti-goat IgA antibodies. Goat IgA is commercially not available. We are trying to get goat IgA purified from commercial firm on payment and then raise anti IgA antibodies.

TABLE VIIIa
ELISA antibody titers in colostrum pools and dried powders with
HRV-4 asimmunising virus (Schedule K)

| Post delivery day | Colostrum | Colostrum Powder | |
|---|---|---|---|
| pool | pool | With Trehalose | Without trehalose |
| 1^{st} Day Colostrum | 1:320000 | 1:160000 | 1:320000 |
| 2^{nd}, 3^{rd} and 4^{th} Day | 1:40,000 | 1:20000 | 1:20000 |
| Colostrum | | | |
| 10^{th} Day Colostrum | 1:5000 | — | 1:1000 |

TABLE VIIIb
“N” antibody titres in colostrum pools and dried powders with
HRV-4 as immunising virus (Schedule K)

| | | | Colostrum Powder | | | |
|---|---|---|---|---|---|---|
| | Colostrum Pool | | With Trehalose | | Without Trehalose | |
| Post delivery | 2 logs | 1 log | 2 logs | 1 log | 2 logs | 1 log |
| day pool | “N” | “N” | “N” | “N” | “N” | “N” |
| 1^{st} day | 4000 | 8000 | 3000 | 5000 | 2000 | 5000 |
| 2^{nd}, 3^{rd}, 4^{th} day | 4000 | 8000 | 100 | — | — | — |
| 10^{th} day | 4000 | 7000 | — | — | — | |

TABLE IXa
ELISA Antibody Titres after Intranasal immunization (Sch. L)

| Post Delivery | Titres in colostrum |
|---|---|
| Day | pools |
| 1^{st} Day | 1:20000 |
| 2^{nd}, 3^{rd}, 4^{th} | — |
| Day | |
| 10^{th} day | — |

TABLE IXb
Neutralising Antibody Titres after Intranasal immunization (Sch. L)

| | Colostrum Pool | |
|---|---|---|
| Post delivery day pool | 2 logs “N” | 1 log “N” |
| 1^{st} day | 500 | 1000 |
| 2^{nd}, 3^{rd}, 4^{th} day | 100 | 100 |
| 10^{th} day | -ve | 100 |

Considering the problems with naturally bred goats, it was again decided to use goats after synchronization and A1, so that the schedule can be planned well

Accordingly, 13 goats were made available for which Synchronization and A1 was carried out at Atpadi.

### EXAMPLE 4

To prepare immune colostrum using semi-purified virus and intramammary route of immunization and to compare results with routine schedule.

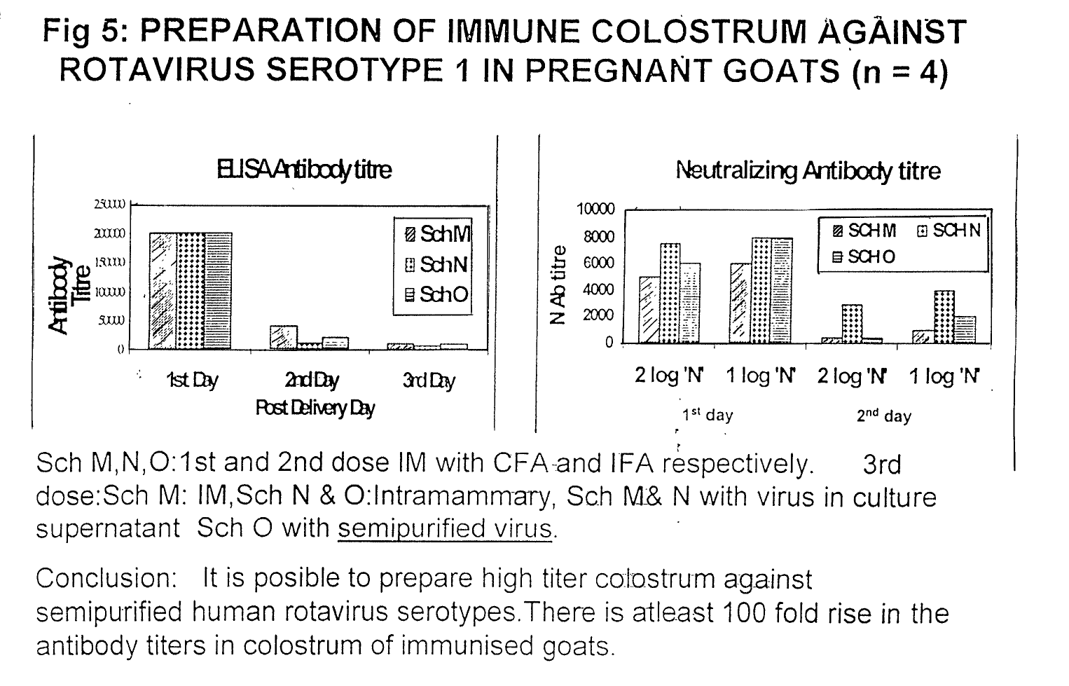

We have used semi purified virus stock which is about 100 fold concentrated for immunization. This is to get rid of all the cellular material of the cell line, employed for the growth of the virus, from the virus preparation. Besides this, it is convenient to carry the small amounts of virus for the field work. Intramammary route of immunization was also used for the first time during the last dose of the virus using schedules viz N & O. The details of the schedule are give in Table X and the results are presented in FIG. 5.

TABLE X
IMMUNISATION SCHEDULE: M, N and O

| | | | | | | |
|---|---|---|---|---|---|---|
| | | | | | | De- |
| | No. | | | | | livery |
| Sched- | of | | | | Route/no. of | Day |
| ule | Goats | Dose | Day | Inoculum | sites | (Avg) |

| | | | | | | |
|---|---|---|---|---|---|---|
| M | 4 | 1 | 0 | HRV-1 + CFA | IM/4 | 32 |
| | | 2 | 21 | HRV-1 + IFA | IM/4 | |
| | | 3 | 30 | HRV-1 + SA-11 | IM/4 | |
| N | 4 | 1 | 0 | HRV-1 + CFA | IM/4 | 31- |
| | | | | | | 36 |
| | | 2 | 21 | HRV-1 + IFA | IM/4 | |
| | | 3 | 30 | HRV-1 + SA-11 | MAMMARY | |
| O | 4 | 1 | 0 | SEMIPURIFIED | IM/4 | 26- |
| | | | | | | 35 |
| | | | | HRV-1 + CFA | | |
| | | 2 | 21 | SEMIPURIFIED | IM/4 | |
| | | | | HRV-1 + IFA | | |
| | | 3 | 30 | SEMIPURIFIED | INTRA | |
| | | | | HRV-1 + SA-11 | MAMMARY | |

Immunization schedules viz. M,N,O were carried out Schedule M was similar to schedule J, because sufficient data could not be obtained with schedule J. Conclusion:

It is clear from the results that the immune colostrum could be prepared employing any one of the schedules viz M,N,O with almost equal efficiency. The neutralizing antibody titers among the pools of immunized goat colostrums, goat sera and kid sera were 1:40, <1:10 and <1:10 respectively. Thus, here is at least 100 fold rise in the antibody titers in the colostruns of immunized goats.

Colostrum obtained from schedule O, in which semipurified virus was used was spray dried as well as freeze dried.

We claim:

1. A process for preparing colostrum to prevent rotavirus induced diarrhoea of G1 to G4 and G5, G6 and G8 to G10 comprising collecting rotavirus specimens from infected children;
mixing the said virus with an adjuvant to obtain water in oil suspension; innoculating said virus+adjuvant mixture by intramuscular route near the lymph nodes of the pregnant goats at multiple sites at different intervals for immunisation; collecting the colostrum from said immunized goats after delivery;
drying the colostrum to obtain dried powder thereof; adding the dried colostrum to a carrier.

2. A process for preparing colostrum to prevent rotavirus induced diarrhoea of G1 to G4 and G5, G6 and G8 to G10 comprising collecting rotavirus specimens from infected children;
mixing the said virus with an adjuvant to obtain water in oil suspension;
innoculating said virus only intramvenous route near the lymph nodes of the pregnant goats at multiple sites at different intervals for immunization;
collecting the colostrum from the immunized goats after delivery; drying the colostrum to obtain dried powder thereof;
adding the dried colostrum to a carrier.

3. A process as claimed in claim 1, wherein in the said step inoculation two liter virus tock is prepared and the same is centrifuged at 5000 rpm for 30 mins to get rid of cell debris and stored at 70° C. till using the stock for immunization.

4. A process as claimed in claim 1, wherein said adjuvant mixture comprises Freund's complete adjuvant heat killed mycobacterium tuberculosis emulsified in mineral oil.

5. A process as claimed in claim 1 wherein said colostrum samples are stored at −20° C.

6. A process as claimed in claim 1 wherein the said step of drying is performed by spray drying the samples.

7. A process as claimed in claim 1 wherein the said colostrum samples are collected on the 1st, 2nd, 3rd and 4th post delivery day and milk on the 10th post delivery day.

8. The colostrum prepared by the process of the preceding claims is used for preventing rotavirus induced diarrhoea in infants and young children.

9. The colostrum as claimed in claim 8 is given to the infants and babies as nutritional supplement.
